(19) United States
(12) Patent Application Publication
(10) Pub. No.: US 2004/0034309 A1
(43) Pub. Date: Feb. 19, 2004

(54) Method and system of defining a model of one or more organs

(75) Inventors: Andrew Pullan, Auckland (NZ); Martin Buist, Auckland (NZ); Leo Cheng, Auckland (NZ)

Correspondence Address: NIXON & VANDERHYE, PC, 1100 N GLEBE ROAD, 8TH FLOOR, ARLINGTON, VA, 22201-4714, US

(73) Assignee: Auckland UniServices Limited

(21) Appl. No.: 10/617,709

(22) Filed: Jul. 14, 2003

(30) Foreign Application Priority Data
Jul. 12, 2002 (NZ) NZ 520136
Apr. 16, 2003 (NZ) NZ 525358

(51) Int. Cl. A61B 5/0402

(52) U.S. Cl. 600/509; 600/513

(57) ABSTRACT

A method of defining a model of one or more organs or part(s) thereof from multiple images of the organ(s) or part(s) thereof, the method comprising the steps of generating a computational mesh of one or more organs or part(s) thereof from multiple images of the organ(s), or part(s) thereof; generating a representation of musculature or part(s) thereof associated with the organ(s); calculating electric and/or magnetic fields associated with the muscle layers; and defining a model based on the computational mesh, and the electric and/or magnetic fields.

### FIELD OF INVENTION

The invention relates to a method and system involving defining a model of one or more organs or part(s) thereof then using the model to interpret remote electrical and/or magnetic recordings. The invention has primary application in data interpretation relating to the gastrointestinal tract. The invention is equally applicable to any other organ from which magnetic or electrical activity can be recorded, for example the heart, brain and uterus.

### BACKGROUND TO INVENTION

Prior art techniques exist to obtain electrical and/or magnetic recordings from gastric activity, from intestine activity, from cardiac activity, and many other musculature organs.

The twelve lead ECG (electrocardiogram) is used to measure cardiac electrical activity and is a standard that has been almost universally adopted. At present, there is no standard for measuring an EGG (electrogastrogram). The low conductivity tissues that lie between the active tissue in the gut and any cutaneous electrodes hinders the recording and subsequent interpretation of EGG signals. The magnetogastrogram (MGG) does not suffer from these problems, as the permeability of biological tissues is very similar to that of free space.

It would be particularly advantageous to create a computer model that is capable of reproducing and/or interpreting MGGs and electrical and/or magnetic recordings of various other body organs.

### SUMMARY OF INVENTION

In broad terms in one form the invention comprises a method of defining a model of one or more organs or part(s) thereof from multiple images of the organ(s) or part(s) thereof, the method comprising the steps of generating a computational mesh of one or more organs from multiple images of the organ(s); generating a representation of musculature associated with the organ(s); calculating electric and/or magnetic fields associated with the muscle layers; and defining a model based on the computational mesh, and the electric and/or magnetic fields.

The invention also comprises a method of estimating the location of one or more sources of magnetic and/or electric fields in a subject by defining a model of one or more organs or part(s) thereof as described above, obtaining one or more measured magnetic and/or electric fields from a subject, and estimating the location of one or more sources of magnetic and/or electric fields based at least partly on the model of one or more organs and the measured magnetic and/or electric fields.

In broad terms in another form the invention comprises a model defining system for defining a model of one or more organs or part(s) thereof from multiple images of the organ(s) or part(s) thereof, the system comprising a mesh generation component configured to generate a computational mesh of one or more organs from multiple images of the organ(s) and a representation of muscle layers associated with the organ(s); an electric/magnetic field component configured to calculate electric and/or magnetic fields associated with the muscle layers; and a model creation component configured to define a model based at least partly on the computational mesh and the electric and/or magnetic fields.

In another form the invention comprises a source location system for estimating the location of one or more sources of magnetic and/or electric fields in a subject.. The system comprises a model defining system as described above and a location estimator configured to estimate the location of one or more sources of magnetic and/or electric fields based at least partly on the model of one or more organs and data obtained from one or more measured sources of magnetic and/or electric fields from a subject.

### BRIEF DESCRIPTION OF THE FIGURES

Preferred forms of the invention will now be described, by way of example, with reference to the accompanying figures in which:

FIG. 1 shows a preferred form method for defining a model in accordance with the invention;

FIG. 2 shows a preferred form system in accordance with the invention;

FIG. 3 illustrates an image of one or more organs of a subject;

FIG. 4 shows a preferred form fitted stomach mesh;

FIG. 5 shows the digitised and fitted outer skin surface of the torso;

FIG. 6 illustrates individual meshes of multiple organs of the digestive system;

FIG. 7 illustrates a representation of musculature;

FIG. 8 shows an example of a high resolution finite difference mesh;

FIG. 9 illustrates the results of a simulation in accordance with the invention;

FIG. 10 illustrates magnetic field vectors generated in accordance with the invention;

FIG. 11 illustrates point source localisation in accordance with the invention;

FIG. 12 shows a sample output generated in accordance with the invention; and

FIG. 13 shows a further sample output generated in accordance with the invention.

### DETAILED DESCRIPTION OF PREFERRED FORMS

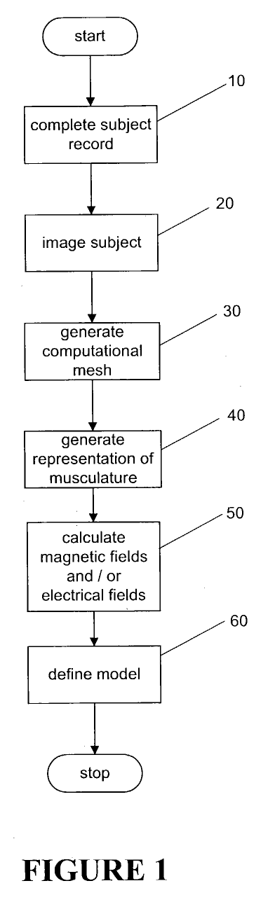

FIG. 1 sets out a preferred form method of the invention for defining a model of one or more organs or parts of organs of a subject. The first step is optionally to complete a subject record 10 containing as much relevant information as possible, including weight, height, age and current medication. The subject record could also include any history of problems associated with the organ(s) of interest, for example digestive problems.

The next step is to obtain images of the subject 20 to obtain the anatomical and geometrical information that is necessary to create a subject-specific model of the digestive or other system and surrounding tissues. The imaging modality used to gain this information on the internal composition of a subject includes one or more of MRI (magnetic resonance imaging), CT (computer tomography), ultrasound, PET (positron emission tomography) and X-ray imaging.

It is also envisaged that the data obtained from the selected imaging modality could be supplemented in one or more ways. For example, an additional imaging technique could be used such as a laser scanner or camera system to capture the external surface of the subject. Distance measurements could be manually taken from the subject, such as lengths and circumferences. A mechanical or other acquisition device could be used to record the location of fiducial markers to aid in the registration of gathered data. Furthermore, the body fat of a subject could be measured.

A contrast agent could be used when imaging the subject such as a barium swallow to better elucidate the geometry of the digestive system, or other technique suitable for the organ(s) of interest.

Following the imaging process, the outputs from the imaging of the subject are likely to be either 2-dimensional image slices that are not restricted to the same orientation, or 3-dimensional volume image sets. These outputs could display one or more organs or one or more parts of organs of the subject imaged.

The information within these images that is of interest for one application of this invention is the location and anatomical configuration of the organs that make up the digestive system. Also of interest are the organs that surround the digestive system and influence the shape or functional properties of the digestive organs.

For each organ or part of interest, representations of the surfaces of the organ are required, as well as any internal surfaces that are required to represent regions with different physiological properties. It is preferable to represent at least the electrically active organs of the digestive system of a subject and all of the surrounding organs that influence magnetic fields and electric fields associated with these electrically active organs. The electrically active digestive organs include the oesophagus or esophagus, the stomach, the small intestine (duodenum, jejunum, ileum) and the large intestine (colon). The tissues of interest surrounding these organs include the liver, the pancreas, the abdominal muscles, the subcutaneous fat, and the fat within the abdominal cavity.

The next step is to generate 30 a computational mesh representing the above features. The generation of the computational mesh is further described below. There are several different ways of creating the computational mesh from multiple images, for example image slices, of the organ or organs.

One such method is to automatically generate computational meshes using software which automatically segments image data and an automated mesh creation process that is subject to constraints depending on the precision requirements of the mesh.

Another method is to create data points from an image set and then fit an initial generic model to the data, minimising the distance between the data and the fitted model. These data points can be created manually using digitising software, for example the software described in patent specification WO 01/01859 to Auckland UniServices Limited. It is envisaged that if the gathered data is too sparse in some areas, perhaps because of an imaging artifact, it is possible to perform a second type of fit known as anthropomorphic fitting. This type of fitting uses a set of fiducial markers to adjust a previously fitted organ mesh so that it matches the current geometry.

In each case, a mesh generation component could be a computer program installed and operating in computer memory which is configured to generate a computational mesh of one or more organs from multiple images of the organ(s).

Following generation of the computational mesh for organs, or parts of organs, the next step is to generate 40 a representation of musculature as will be described below. The musculature generating the magnetic fields are either inside or form part of the organs. IS There are also muscles outside the organs that act in a passive manner, such as the abdominal wall muscles, but these are not a primary field source. The mesh generation component could also be configured to generate representations of the muscle layers associated with the organ(s), either inside or forming part of the organ(s).

It is often difficult to image a subject in sufficient detail to obtain all the required information, and so it is preferable to add prior knowledge to the anatomically accurate geometrical model. This information could include data on the cellular and tissue composition of each organ and the spatial and temporal variations that occur. In muscular regions, the microstructure of the muscles needs to be added as preferential directions of electrical propagation and contraction are often present.

A combination of accurate geometry and the appropriate physiology contributes to the generation of a specific subject model of the system of interest. This in turn provides a framework in which the equations governing the processes representing gastric activity, intestinal activity, cardiac activity, or other activity can be solved accurately.

Referring to FIG. 1, the next step is to calculate 50 the magnetic fields associated with the model layers generated in steps 30 and 40 and to define 60 a model based on the computational mesh, the representation of musculature, and the electric and magnetic fields.

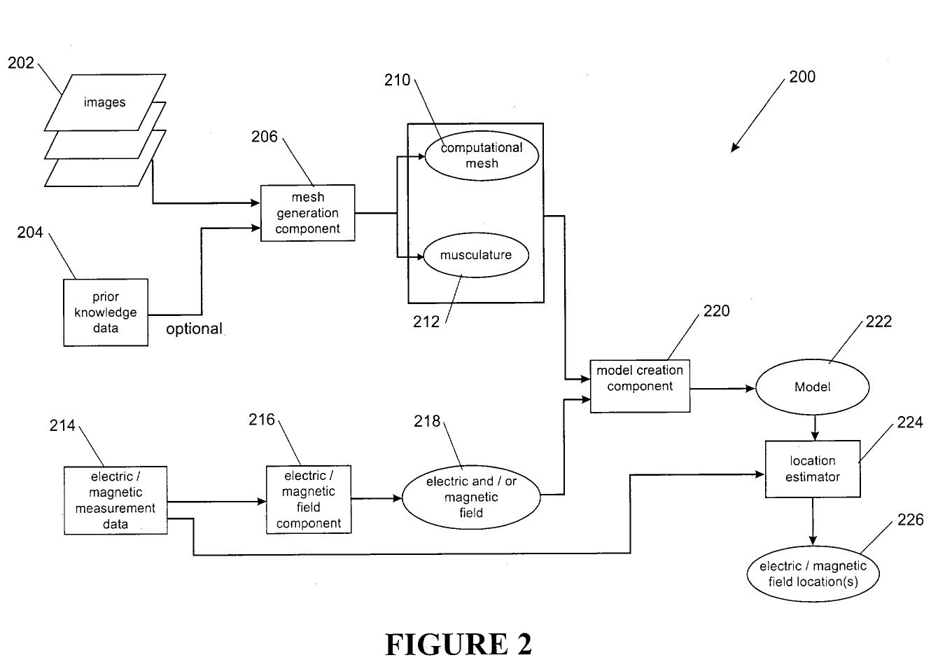

FIG. 2 illustrates a preferred form system 200 for carrying out the method described above. Images 202 represent, for example, multiple image slices of an organ or organs. Images 202 and optionally prior knowledge data 204 is input to mesh generation component 206. The mesh generation component 206 is configured to generate a computational mesh 210 and optionally representations of the muscle layers 212.

As will be described below, it is envisaged that data obtained from non-invasive electric/magnetic measurement data 214 be used as input to an electric/magnetic field component 216. The electric/magnetic field component 216 could include a computer program in which mathematical models and related mathematical equations are calculated. The component 216 is configured to calculate electric and/or magnetic fields 218 associated with muscle layers.

The computational mesh 210, the electric and/or magnetic fields 218 and optionally the muscle layer representations 212 are then input to a model creation component 220. The model creation component is preferably a software-based component configured to define a model based on the above inputs. Model 222 preferably represents one or more organs or part or parts thereof.

The system 200 optionally further includes a location estimator 224. The estimator 224 preferably comprises a computer program that is configured to solve an inverse problem. The estimator 224 takes as input the model 222 generated by component 220 and electric/magnetic measurement data 214 and estimates the location of one or more sources of magnetic and/or electric fields shown in FIG. 2 as electric/magnetic field location(s) 226.

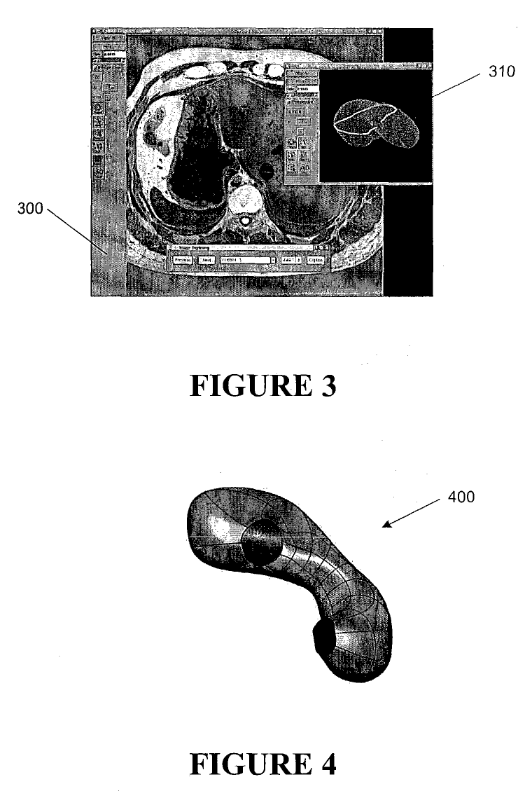

FIG. 3 illustrates an image 100 of one or more organs of a subject. Photographic images in the axial plane of a subject that are available at 1 mm intervals over the length of the subject body have been digitised. The components of interest are traced on every second transverse digital human image which translates to a vertical resolution of 2 mm as the original slices were taken at 1 mm intervals.

The components particularly of interest in gastrointestinal activity are the oesophagus, the stomach, the duodenum, the jejunum and the ileum. The outer surface of the oesophagus, stomach, duodenum and small intestine are traced, and the centre line of the remainder of the small intestine is also located. It will be appreciated that the components of interest will vary depending on the activity under analysis.

For the oesophagus, stomach and duodenum, initial linear quadrilateral surface elements are created by selecting a regular array of data points to be the initial nodal positions. From this, the nodal positions and then nodal derivatives are fitted to the digitised data set for each component to create a bi-cubic Hermite outer surface description of the system.

The RMS (root mean square) errors between the final fitted surfaces and the digitised data are less than 1 mm for each of the meshes. From the outer surface, a volume mesh is created through an inward cylindrical projection and a wall thickness of 5 mm is chosen as an average value.

In FIG. 3 the outer wall of the stomach has been digitised. Graphics window display 300 shows the currently digitised points in 3-dimensions and provides an indication of where data may be sparse, missing or incorrectly placed. An image control dialog box 310 enables the manipulation of an image set and also allows multiple organs to be digitised into separate data groups on a single image. Once the data has been collected, the initial computational mesh is generated to which the data is fitted.

FIG. 4 illustrates at 400 a preferred form fitted stomach computational mesh which is the result of the fitting procedure to the outer wall of the stomach. A C¹ continuous quadrilateral description of the surface is calculated which is then used to generate each of the individual points visible on the surface. The average error between the data points and the surface projection of these data points on the mesh is approximately 0.6 mm.

A volume computational mesh can also be generated by either applying the same digitising and fitting process to the inner surface of the stomach, or by projecting the outer surface inwards based on known information about the thickness of the stomach wall.

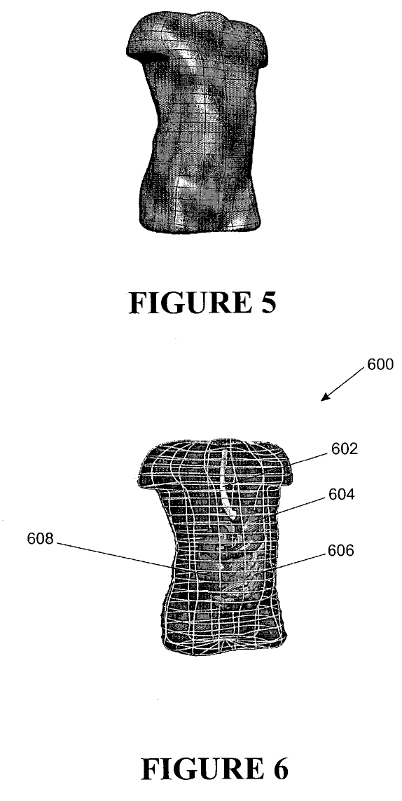

It is also envisaged that the outer skin surface of the torso be digitised and fitted as shown in FIG. 5. As all the data is referenced to a common origin, the position of the organs within the torso is consistent with the original images.

As shown in FIG. 6, individual meshes of multiple organs of the digestive system can be generated and combined with a mesh of the outer skin surface of the torso. The combined computational mesh 600 could include the oesophagus 602, the stomach 604, the duodenum, jejunum and ileum making up the small intestine 606 and the colon or large intestine 608. The sigmoid colon has been omitted from this image for clarity.

The centre line of the jejunum and ileum is used to create a topologically cylindrical mesh through a radial projection. The outer diameter of the jejunum is 40 mm with a wall thickness of 4 mm and the diameter of the ileum is 37.5 mm with a wall thickness of 3 mm. The transition from the jejunum to the ileum is performed on a length basis as the jejunum occupies approximately 40% of the length of the small intestine whereas the ileum occupies approximately 60% of the length.

It is envisaged that all the above mesh components can be customised through a host mesh fitting technique to individual subjects to account for subject variability.

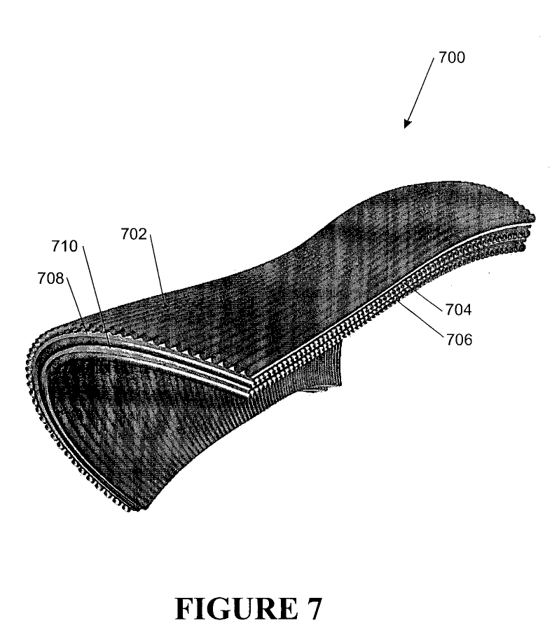

FIG. 7 shows a representation 700 or model of the musculature in a small section of the stomach wall. The representation shows longitudinal muscle layer 702, circular muscle layers 704 and 706, and interstitial cells of Cajal 708 and 710.

Once the combined computational mesh is defined, and representation of musculature generated, it is then necessary to determine the electrical activity within the organ(s) or part(s) thereof, for example the stomach and intestine and the related muscle layers, which is producing a known or measured magnetic or potential field on or near the torso surface. Sensors can be placed near or on the torso to obtain non-invasive measurements about the electrical or magnetic fields generated from within the torso. Electrical fields can be measured in a non-invasive manner by electrodes placed directly on the skin surface.

Known electrical mapping systems are able to record from one to many hundred electrodes simultaneously. These mapping systems have been typically designed for recording electrical activity from the heart or brain. These measurements are obtained relative to a single or combination of electrodes recorded at the same time.

Magnetic sensors are capable of recording changes in magnetic fields without direct contact with the torso surface. These fields can be recorded using recording devices known as SQUIDS, for example as described in U.S. Pat. specification No. 5,771,894 to Richards et al.

The locations on which these electrodes or sensors are positioned relative to the torso are also critical for subsequent measurement. As electrodes are placed directly on the skin surface, the relative positions of these electrodes will shift for each subject. On the other hand magnetic sensors are non-contact and are typically in a fixed position relative to each other. This means that their relative positions only need to be determined once but their overall positions relative to the torso must be determined each time.

The EGG (electrogastrogram) and the MGG (magnetogastrogram) record not only the surface projections of the summated action potentials of gastric smooth muscles, but also a compound signal from different electrical sources in the torso. These magnetic and potential fields are attenuated and filtered by the surrounding organs, for example muscle and fat layers. The field data can be interpreted by pattern matching against known signal sets, or by using a mathematical model to interpret the data directly.

The process of pattern matching, or comparing signal traces between a health normal and an abnormal patient is commonly used to determine the presence of an abnormality. For such a process to be a success, the person interpreting the data needs to take into account many factors, including the size of the subject, location and relative placement of sensors or electrodes, age and so on. It is assumed that the sensors or electrodes are placed at standard locations so that results can be compared between patients. The process can often be flawed and require significant training and an established and consistent database for comparison of results.

An alternative method of interpreting the data is through use of a mathematical model where measured data can be used to directly interpret the field data. The use of a mathematical model makes such an interpretation less subjective to human judgement, as results are typically displayed in a more direct and meaningful manner closely related to the underlying origins of the events. As described above, electric/magnetic field component could include a computer program in which the mathematical model and related mathematical equations are calculated. The model creation component is configured to define a model based on the computational mesh, the magnetic fields calculated by the following equations and/or the representation of muscle layers.

Assumptions are made as to the equations that govern the electric and magnetic activity in the subject and these equations are then solved once the computational mesh has been created.

The equations that govern all magnetic and electric fields are known as Maxwell's equations. The frequencies of the biological signals that are of interest are generally less than 100 Hz, and the magnetic permeability of biological tissue is very similar to that of air. On this basis, Maxwell's equations may be simplified to what is termed the quasi-static assumption.

The governing equations in differential form can be written as:

$$∇ \cdot E = \frac{ρ}{{ϵ}_{0}} \qquad ( 1 )$$

∇×E=0 (2)

∇·B=0 (3)

∇×B=μ₀J (4)

E is the electric field intensity, B is the magnetic flux density, J is the electric current density, ρ is the electric charge density, ε₀ is the permittivity of free space, and μₒ is the permeability of free space.

In addition to the above equations, the continuity equation is needed to ensure there is no build up of electric charge within a region:

∇·J=0 (5)

Three main formulations arise from the above equations. The first formulation is a set of equations known as the bidomain equations that govern the spread of electrical activity within excitable tissue. The second formulation is a generalised Laplace equation that describes the current flows within passive tissue regions. The third formulation is an equation which takes the electric fields as input and then calculates the magnetic field generated by the electrical activity.

The first formulation of equations model active tissue as two inter-penetrating domains that occupy the same physical space. The relationship between potentials in the two spaces across the cell membrane is the first equation in the bidomain system.

Vₘ=φᵢ−φₑ (6)

The intracellular domain represented as ᵢ represents the interior of the cells, and the extracellular domain represented as ₑ represents the material surrounding the cells. Other terms contain ₘ to indicate they are properties of the cell membrane.

The two bidomain equations are:

$$∇ \cdot ( {σ}_{i} ∇ {V}_{m} ) + ∇ \cdot ( {σ}_{i} ∇ {ϕ}_{e} ) = {A}_{m} ( {C}_{m} \frac{\partial {V}_{m}}{\partial t} + {I}_{ion} ) + {I}_{s 1} \qquad ( 7 )$$

∇·((σᵢ+σₑ)∇φₑ)=−∇(σᵢ∇Vₘ)−Iₛ₂ (8)

Here the σ terms are tissue conductivities which in general will be tensors, the φ terms are potentials, the Vₘ term is the transmembrane potential, the potential difference across the cell membrane, Aₘ is the surface to volume ratio of the membrane and Cₘ is the membrane capacitance. Individual cellular models are able to plug directly into these equations through the lᵢₒₙ term in the first equation.

At a fine scale, each cellular model is able to incorporate complex subcellular processes. Externally applied currents may be injected into either domain through Iₛ₁ or Iₛ₂. These equations are solved using either the finite element-based finite difference method or the structured finite element method.

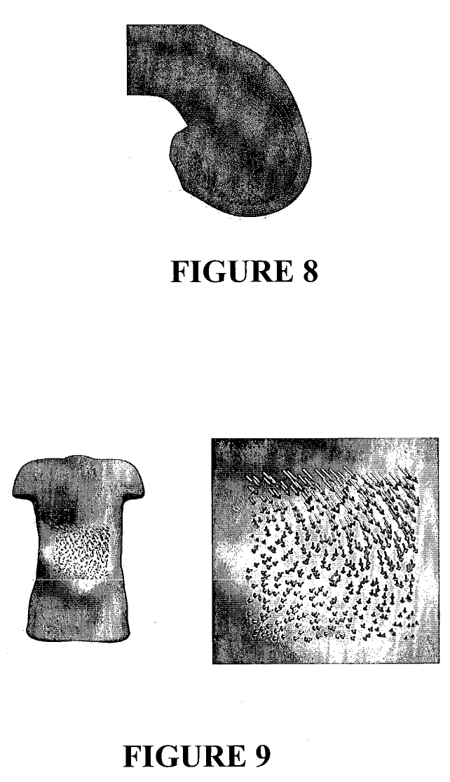

FIG. 8 shows an example of a high resolution finite difference mesh created over the finite elements of the stomach using the first formulation.

The second formulation that arises from Maxwell's equations is a generalised Laplace equation:

∇·(σₒ∇φₒ)=0 (9)

The o subscripts denote quantities outside the active region. Bidomain equation (8) that solves for φₑ is directly coupled to the external passive regions through the interface conditions on shared boundaries:

φₑ=φ₀ (10)

σₑ∇φₑ·nₑ=σₒ∇φₒ·nₒ (11)

Similar formulae apply to interfaces between passive regions, ensuring that the potential fields and current flows are properly conserved. For electrically isotropic regions, the conductivity reduces to a single value and the boundary element method is used to solve these equations. For electrically anisotropic regions the conductivity is a 3×3 tensor and the finite element method is used.

The third formulation defines the magnetic field generated by the electrical activity and is defined in terms of the curl of a vector field A:

B=∇×A (12)

The vector potential field A is then defined in terms of the electric current density:

∇²A=−μ₀J (13)

The current density comprises two components. The first component is the contribution of the primary current sources, in this case a function of the transmembrane potential gradient. The second component is the contribution from distribution of the resistive network within the torso volume, or other relevant body volume:

J=J^{p}−σ∇φ (14)

The above equations are solved using the finite element and boundary element meshes that are used to model the passive electric fields.

A typical solution consists of four sets of calculations that must be performed at each time step. In the first of these calculations, the cellular terms are updated throughout the active region. In the second set of calculations, the transmembrane potential is calculated from the cellular terms and known diffusive properties. In the third set of calculations, the coupled extracellular/passive torso problem is solved to create a continuous electric field throughout the torso. In the fourth set of calculations, this electric field is used as an input to the calculation of the magnetic field.

A simplification to this coupled system involves the introduction of equivalent dipolar source terms to represent the contribution of the active region. In this case, the active region is solved in isolation and equivalent sources are calculated through the vector summation of the primary cellular sources. Typically, this process produces in the order of tens of dipole sources to represent the electrical activity. These sources are then placed into the passive torso model and the appropriate equations are solved to obtain the electric and magnetic fields within and surrounding the torso.

FIG. 9 illustrates the results of a simulation in accordance with the invention. The bulk of the electrical activity is located halfway along the duodenum at the beginning of the small intestine within the torso. From this cellular activity, equivalent dipole sources are used as inputs to the passive torso solution that generates the electric fields throughout the torso. The primary sources and these electric fields are then used to generate the magnetic field just outside the surface of the torso. In the figure, the magnetic field is drawn as arrows that have been seeded at random points within the square in space. Each arrow points in the direction of the calculated magnetic field and the length of the arrow indicates the strength of the field.

The invention is particularly suited to solving an inverse problem. An inverse problem is a general term for a class of problems which attempt to determine the sources or events which produce a known result. Mathematically they can be written in the general form Aₓ=b where x is an unknown variable, b is a known or measured variable and A is a function which is capable of mapping between the two variables.

The inverse problem for the stomach and intestinal system involves determining the electrical activity within the stomach and intestine which is producing a known or measured magnetic or potential field on or near the torso surface. In the case of the above equation, x represents the unknown electrical sources in the stomach and/or intestine, b represents a known or measured resultant field (external electrical potential or magnetic fields) while A is a function which relates two fields which takes into account the geometries of the organs in the torso and their electrical conductivities which effect the path and pattern of the electrical/magnetic activity as it disperses from the site of activation.

Inverse problems also fall into a class of problems which are known as ill-posed. This means that small amounts of error in the solution process and input data can result in the large and disproportionate errors in the computed solution. This means they are mathematically difficult to solve.

The use of a mathematical model makes interpretation of data less subjective to human judgement as the results are typically displayed in a more direct and meaningful manner closely related to the underlying origins of the events.

Inverse algorithms are used to compute a “source” given a known geometry and surrounding potential or magnetic field. Such a source is usually a simplified, at a spatial scale larger than that of a cell, but realistic representation of the underlying events actually occurring in the body.

Point source inverses locate dipoles at sites of interest. Dipole sources are a mathematical way of representing the strength and direction of a field with only a few parameters. A dipole is essentially a vector quantity and defined by six components. Three components define the centre for example (x, y, z) and three components define the orientation for example (dz, dy, dz) assuming a rectangular Cartesian space.

Dipoles are commonly used in detecting events and localising regions of interest in the brain from both electrical and magnetic fields and used to a lesser extent with electrical fields for imaging the heart.

A non-linear optimiser is used which minimises the difference between a known/measured potential/magnetic field and that computed from an estimated source applied within the torso by adjusting the source parameters. Thus, from an arbitrary initial estimate of a dipole(s), the resultant potential/magnetic fields are computed, and then sequentially adjusted until the potential/magnetic fields match. These can be described mathematically as:

Minimise F=f(B,B′)+lambda f(Phi, Phi′) w.r.t. dipole parameters (15)

Where B and B′ are the known and computed magnetic field intensity, Phi and Phi′ are known and computed electrical potentials, lambda is a scaling factor to provide a weighting between the two objectives, and f is a general function which provides a measure of the difference between the two fields, for example absolute magnitude difference, difference in pattern, difference in relative timings.

Distributed source inverse algorithms are commonly used in solving the inverse problem of electrocardiography, although point source inverses are also used to a lesser degree.

There are two main source formulations, that of a potential-based inverse and that of activation time inverses. The potential-based formulation determines a temporally varying electrical potential field for a given surface, while an activation time inverse algorithm determines the time at which the electrical potential wave front passes each point in space.

Having obtained magnetic recordings from a subject and devised an accurate geometric model of the subject, the invention permits localisation of the source of the magnetic field. Magnetic fields are typically recorded at between 20 and 100 sites and provide a combination of gradient magnetic vector field and absolute magnetic field vector recordings.

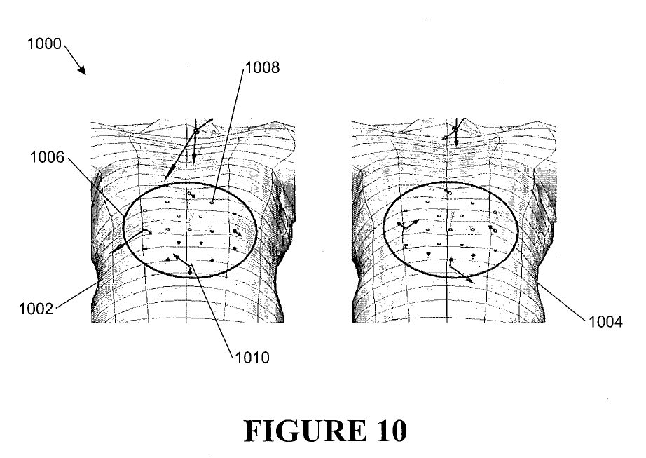

FIG. 10 illustrates magnetic field vectors 1000 that are recorded from a human subject shown at 1002 and 1004 using a SQUID 25 seconds apart. The magnetic fields represent a summation of the electrical activity occurring within the stomach indicated generally at 1006. Images 1002 and 1004 show 37 vector magnetic/gradient field recordings at 20 physical locations just above the body surface. One such physical location is indicated at 1008. At five of the locations, there are multiple channels recording at different vector directions, for example the location indicated at 1010.

Using simulation studies and under controlled conditions, it is possible to localise the source of electrical activity to less than 1 mm. The localisation is performed using both electrical recordings and magnetic recordings, and a combination of the two as the “measured” field.

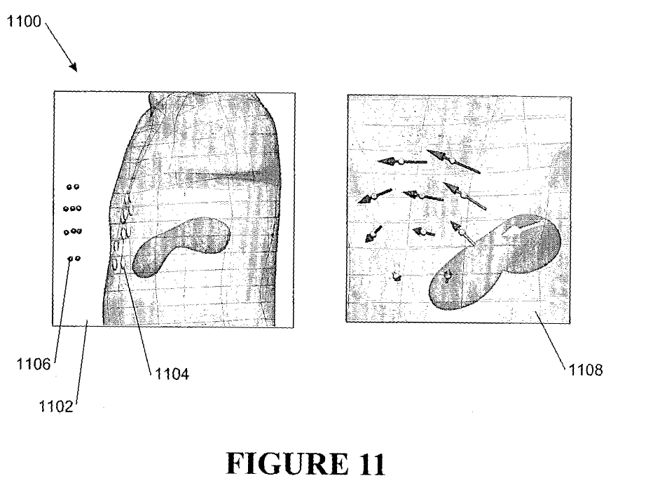

FIG. 11 illustrates point source localisation 1100 of a site of focal activity in the stomach below the fundus. Recording electrodes are shown in 1102, for example at 1104 and magnetic sensors near the skin surface indicated at 1106.

In diagram 1108, magnetic fields are used to determine the site of focal activity on the stomach. In this case the dipole centre has been localised to within 1 mm under controlled conditions.

The model defined in accordance with the invention has the potential to provide valuable insight to activity which cannot be easily observed or measured using existing imaging methods. Validation of the model can be conducted using analytic models or validation experiments. Analytic models usually involve simplified geometries and known initial and boundary conditions are applied to obtain a known solution. A more thorough test involves the use of validation experiments where measurements are obtained both internal and external of the subject. In this way, the computed solutions can be compared to those measured directly within the torso.

One of the areas of application for the invention is in the modelling of gastroparesis and gastric uncoupling. One type of gastroparesis can occur when nerves to the stomach are damaged or stop working. The vagus nerve controls the movement of food through the digestive tract. If the vagus nerve is damaged, the muscles of the stomach and intestines do not work normally, and the movement of food is slowed or stopped.

Gastroparesis is relatively prevalent in patients with type 1 diabetes and is detrimental to the patient's health as the delay in gastric emptying inhibits the control of blood glucose levels. At least 20% of people with type 1 diabetes develop gastroparesis. This condition also occurs in people with type 2 diabetes although with less frequency.

The cause of gastric uncoupling can be mechanical where external intervention, for example surgery or trauma, creates an electrical break in the stomach wall. The normal pacemaking wave is no longer able to propagate past the site of the break and pacemaker cells distal to the break begin pacing the distal stomach at a slower rate than the normal dominant frequency.

Using the invention, a model is created of a slice through the stomach wall along the greater curvature. The model includes Interstitial Cells of Cajal (ICCs) along with circular and longitudinal smooth muscle layers.

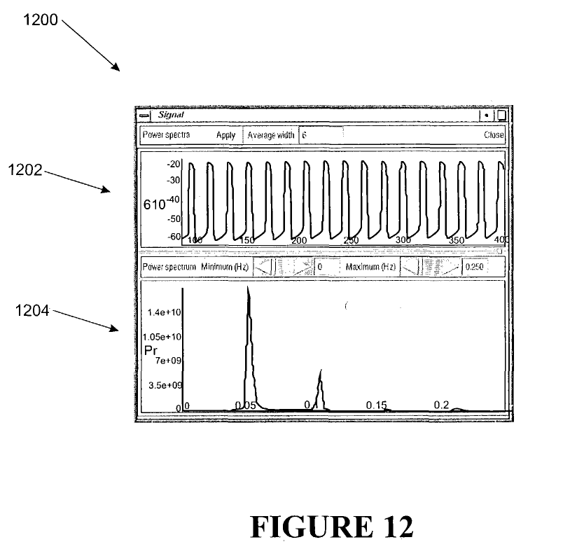

The model is first executed in the absence of any mechanical uncoupling. In this situation, the distal regions of the tissue model were entrained at the frequency of the dominant pacemaker which was approximately 3.06 cpm (0.01 Hz). Transmembrane potentials and the power spectrum of the electrical control activity (ECA) over time are displayed in the signal output window in FIG. 12.

The sample output 1200 shows at 1202 the computed transmembrane potentials in mV and time in seconds and shows at 1204 the power spectrum of the top trace where the frequency is in Hz.

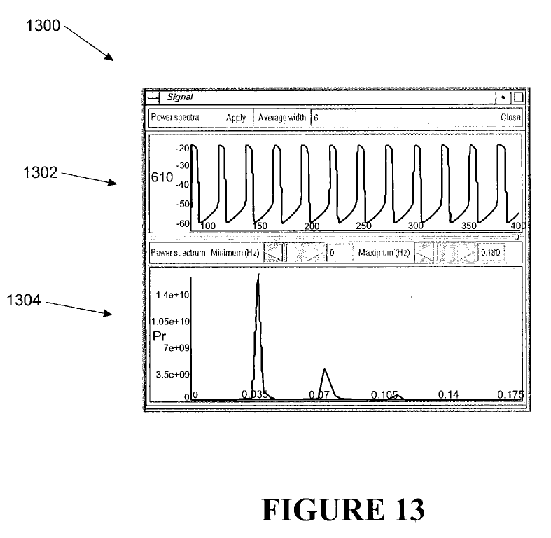

A conduction blockage was then introduced 50% of the way from the dominant pacemaker site to the terminal antrum. Proximal to the blockage, the dominant ECA frequency remained unchanged, but distal to the blockage the dominant frequency was reduced to 2.22 cpm (0.038 Hz) as shown in FIG. 13.

FIG. 13 shows at 1300 sample output from the distal region of the tissue model after mechanical uncoupling. Graph 1302 shows the transmembrane potential in mV plotted against time in seconds and 1304 shows the power spectrum of the signal trace with a decreased dominant frequency shown in Hz.

This bradygastria due to mechanical uncoupling coincides well with measured bradygastrias of the same origin.

The examples described above with reference to FIGS. 3 to 12 have particular application in data interpretation relating to the gastrointestinal tract. It will be appreciated that the same techniques could be applied to data interpretation related to gastric, intestine, and cardiac activity, and could be used to model and analyse any organ, combination of organs, or part(s) thereof.

The foregoing describes the invention including preferred forms thereof. Alterations and modifications as will be obvious to those skilled in the art are intended to be incorporated within the scope hereof, as defined by the accompanying claims.

1. A method of defining a model of one or more organs or part(s) thereof from multiple images of the organ(s) or part(s) thereof, the method comprising the steps of:
generating a computational mesh of one or more organs or part(s) thereof from multiple images of the organ(s), or part(s) thereof;
generating a representation of musculature or part(s) thereof associated with the organ(s);
calculating electric and/or magnetic fields associated with the muscle layers; and
defining a model based on the computational mesh, and the electric and/or magnetic fields.

2. A method as claimed in claim 1 further comprising the steps of:
obtaining non-invasive measurements of electrical and/or magnetic activity from a subject; and
defining the model based at least partly on the measured activity.

3. A method as claimed in claim 2 further comprising the steps of:
estimating one or more sources of electrical and/or magnetic activity within a subject; and
defining the model based at least partly on differences between the estimated sources and the measured activity.

4. A method as claimed in claim 3 wherein the estimated and/or measured activity is associated with gastric activity.

5. A method as claimed in claim 3 wherein the estimated and/or measured activity is associated with intestinal activity.

6. A method as claimed in claim 3 wherein the estimated and/or measured activity is associated with cardiac activity.

7. A method of estimating the location of one or more sources of magnetic and/or electric fields in a subject comprising the step of:
defining a model of one or more organs or part(s) thereof by the method as claimed in claim 1;
obtaining one or more measured sources of magnetic and/or electric fields from a subject; and
estimating the location of one or more sources of magnetic and/or electric fields based at least partly on the model of one or more organs and the measured sources of magnetic and/or electric fields.

8. A model defining system for defining a model of one or more organs or part(s) thereof from multiple images of the organ(s) or part(s) thereof, the system comprising:
a mesh generation component configured to generate a computational mesh of one or more organs or part(s) thereof from multiple images of the organ(s) or part(s) thereof and a representation of musculature or part thereof associated with the organ(s);
an electric/magnetic field component configured to calculate electric and/or magnetic fields associated with the musculature; and
a model creation component configured to define a model based at least partly on the computational mesh and the electric and/or magnetic fields.

9. A model defining system as claimed in claim 8 wherein the model creation component is configured to define the model based at least partly on measured activity data obtained from non-invasive measurements of electrical and/or magnetic activity from a subject.

10. A model defining system as claimed in claim 9 wherein the model creation component is configured to define the model based at least partly on differences between the measured activity data and estimated activity data obtained from estimating one or more sources of electrical and/or magnetic activity within the subject.

11. A model defining system as claimed in claim 10 wherein the measured activity data and/or estimated activity data is associated with gastric activity.

12. A model defining system as claimed in claim 10 wherein the measured activity data and/or estimated activity data is associated with intestine activity.

13. A model defining system as claimed in claim 10 wherein the measured activity data and/or estimated activity data is associated with cardiac activity.

14. A source location system for estimating the location of one or more sources of magnetic and/or electric fields in a subject, the system comprising:
a model defining system as claimed in claim 8; and
a location estimator configured to estimate the location of one or more sources of magnetic and/or electric fields based at least partly on the model of one or more organs and data obtained from one or more measured sources of magnetic and/or electric fields from a subject.
